(19) United States
(12) Patent Application Publication
(10) Pub. No.: US 2004/0115137 A1
(43) Pub. Date: Jun. 17, 2004

(54) Water-soluble film for oral administration

(76) Inventors: Andrew P. Verrall, Crown Point, IN (US); P. Scott Bening, Crown Point, IN (US); Karen A. Kugler, Porter, IN (US)

Correspondence Address: ROBERT W. DIEHL, 311 S. WACKER DRIVE, 53RD FLOOR, CHICAGO, IL, 60606-6622, US

(21) Appl. No.: 10/371,731

(22) Filed: Feb. 21, 2003

Related U.S. Application Data
(60) Provisional application No. 60/434,089, filed on Dec. 17, 2002.

(51) Int. Cl. A61K 9/68; A61K 7/16

(52) U.S. Cl. 424/48; 424/49

(57) ABSTRACT

Physiologically acceptable films, such as water-soluble films, are disclosed. The films include a water-soluble film-forming polymer such as methylhydroxypropylcellulose and/or sodium alginate. Edible films are disclosed that include methylhydroxypropylcellulose and/or sodium alginate, emulsifier, breath freshening agents, stabilizing agents, plasticizers, surfactants, disintegrants, and preservatives. The edible films may be used to deliver an effective amount of an agent for killing bacteria that causes such maladies as dental plaque, gingivitis, bad breath, or the like. The film may optionally contain pharmaceutically active agents.

### RELATED APPLICATIONS

The present application claims the filing benefit of U.S. Provisional Application No. 60/434,089, filed Dec. 17, 2002.

### TECHNICAL FIELD OF INVENTION

The present invention relates generally to the field of water-soluble film, and particularly to such film used to carry a therapeutic or cosmetic agent for oral administration.

### BACKGROUND OF THE INVENTION

As society has evolved, people have become significantly busier in their daily routine and have come to accept some conditions as a result. One of these conditions is decreased oral hygiene. While most people make a point of maintaining oral hygiene when they are at home, many have forgotten the importance of this daily requirement while at work or on the go.

There are a plethora of excuses as to why a person might not engage in maintaining adequate oral hygiene at work or in public. For example, the devices many use for maintaining oral hygiene are not convenient to use at work or where bathroom facilities are scarce. These devices are typically designed to be used in the privacy of one's home.

The typical methods of oral cleansing and hygiene include brushing, flossing, gargling, and tongue cleansing. These methods are not always acceptable depending upon the circumstances, such as during a business meeting. Other less impolite products have been developed such as breath freshening gums and lozenges. Some problems that exist with using these products as part of a method of breath freshening is the disruption caused by chewing or sucking on the substance as well as the time required for the substance to have a significant affect.

Similar to the above mentioned products, mouthsprays may be utilized as a portable method of breath freshening. This product's use as a method of breath freshening has the benefit of strong breath freshening action with the drawbacks of noisy operation that draws attention to the user as well as the expensive packaging that must be utilized for storage. Not only is the product noisy to administer as well as costly to package, but it has the potential to irritate the user's eyes as well as stain his or her clothes if not properly administered.

As a solution to these problems, the present invention has been developed which provides a physiologically acceptable film which adheres to and rapidly dissolves within the mouth of a consumer. Furthermore, the invention provides improved methods of treating halitosis, administering a medicament through a mucous membrane, treating xerostomia, and treating plaque or gingivitis.

### SUMMARY OF THE INVENTION

Generally, the present invention comprises a water-soluble film composition for oral administration having as components thereof primary film forming agents of from about 0 to about 70%, emulsifier from about 0 to about 20%, stabilizing agent from about 0 to about 25%, mouthfeel enhancer from about 0 to about 25%, plasticizer from about 0 to about 15%, surfactant from about 1 to about 5%, sweetener from about 0.5 to about 5%, disintegrant from about 1 to about 5%, salivating agent from about 1 to about 5%, flavoring agent from about 1 to about 40%, and optional coloring agent.

Specifically, as one embodiment of the present invention, a water-soluble film comprising methylhydroxypropylcellulose, gum arabic, microcrystalline cellulose, glycerin, Polysorbate 80, sorbitol, sucralose, cross-linked carboxymethylcellulose, flavoring agents, potassium sorbate, sodium benzoate, and coloring agents is disclosed. In one embodiment of the invention the film comprises up to 70% methylhydroxypropylcellulose, up to 20% gum arabic, up to 25% microcrystalline cellulose, up to 15% glycerin, up to 5% Polysorbate 80, up to 15% sorbitol, up to 5% sucralose, up to 5% cross-linked carboxymethylcellulose, up to 40% flavoring agents, up to 0.2% potassium sorbate, up to 0.2% sodium benzoate and coloring agents. An alternate embodiment of the invention may comprise up to 70% sodium alginate, with or without the methylhydroxypropylcellulose.

A method of delivering an oral care agent to the oral cavity is also generally disclosed. A specific method of the invention comprises the steps of providing a water-soluble film having as primary film formers methylhydroxypropylcellulose and/or sodium alginate, introducing the film into the oral cavity of a user, and allowing the film to dissolve in the oral cavity of the user wherein the one or more primary film formers prolong the efficacy of a breath freshener or other essential.

These and other features are provided in the present invention. A more detailed description of the several components, their purposes, and possible alternative embodiments are set forth in the detailed discussion following.

### DETAILED DESCRIPTION OF THE INVENTION

While the present invention is susceptible of embodiment in many different forms, this disclosure will describe in detail at least one preferred embodiment, and possible alternative embodiments, of the invention with the understanding that the present disclosure is to be considered merely as an exemplification of the principles of the invention and is not intended to limit the broad aspect of the invention to the specific embodiments illustrated.

Further, it should be known that the ranges provided herein are percentages by weight of the composition. It should also be understood that all disclosed ranges, claimed or otherwise, include all combinations of sub-ranges which fall within any such described range.

The present invention provides a single layer, water-soluble film composition, as well as methods of using the same. The film is to be administered orally where, upon disintegration and dissolution, an effective amount of a therapeutic or cosmetic agent may be released and simultaneously absorbed by the buccal cavity of the user.

In an embodiment of the present invention the composition includes an effective amount of at least one primary film forming agent, an effective amount of at least one stabilizing agent, an effective amount of an emulsifier, an effective amount of a disintegrant, and an effective amount of at least one plasticizing agent.

The film forming agent provides structure to the film of the present invention. The effective amount of the film forming agent ranges from about 10% to about 90%, more preferably about 30% to about 70% by weight of the composition. Film forming agents that can be utilized with the water-soluble film composition of the present invention include, but are not limited to, cellulose ethers, modified starches, natural gums, edible polymers, seaweed extracts, land plant extracts, pullulan, derivatives thereof and combinations thereof. It should be understood by those skilled in the art that other film forming agents can be utilized which possess the desirable film properties of the present invention.

Examples of cellulose ethers include, but are not limited to, methylhydroxycellulose, methylcellulose, ethylcellulose, hydroxyethylcellulose, carboxymethylcellulose, derivatives thereof and combinations thereof. Modified starches include, but are not limited to, acid and enzyme hydrolyzed corn and potato starches, derivatives thereof and combinations thereof. Additionally, examples of natural gums include, but are not limited to, gum arabic, guar gum, locust bean gum, carrageenan gum, karaya, ghatti, tragacanth agar, tamarind gum, xanthan gum, derivatives thereof and combinations thereof. Examples of edible polymers that can be utilized in this invention include, but are not limited to, microcrystalline cellulose, cellulose ethers, xanthan, derivatives thereof and combinations thereof. Seaweed extract examples include, but are not limited to, sodium alginate, carrageenans, derivatives thereof and combinations thereof. Land plant extracts include, but are not limited to, konjac, pectin, arabinoglactan, derivatives thereof and combinations thereof. It should be understood by those skilled in the art that other cellulose ethers can be utilized which possess the desirable film properties of the present invention.

The preferred film forming agents of the present invention are sodium alginate and methylhydroxypropylcellulose (MHPC). It should be understood by those skilled in the art that other edible film forming agents can be utilized which possess the desirable film properties of the present invention.

The emulsifier of the present invention acts as an oil-in-water emulsifier. Emulsifiers suitable for use in the edible film of the present invention include, but are not limited to, gum arabic, gum karaya, polyoxyethylene sorbitan esters, e.g., Polysorbate 80, lecithins, mono- and diglycerides, propylene glycol monoesters, polyglycerol esters, sucrose esters, succinylated esters, derivatives thereof and combinations thereof. They can be utilized in amounts ranging from about 0% to about 40%, more preferably about 0% to about 25% by weight of the composition. The preferred emulsifiers for use in the edible film of the present invention are gum arabic, which also functions as a flavor fixative and water binder, and Polysorbate 80. It should be understood by those skilled in the art that other emulsifiers can be utilized which possess the desirable film properties of the present inventions.

The stabilizer agent of the present invention is utilized primarily as an emulsion stabilizer. The effective amount of the stabilizer agents range from about 0% to about 45%, more preferably about 4% to about 25% by weight of the composition. Examples of suitable stabilizing agents of the present invention include, but are not limited to, gum arabic, microcrystalline cellulose, carrageenan, xanthan gum, locust bean gum, derivatives thereof and combinations thereof. The preferred stabilizing agents of the present invention are gum arabic and microcrystalline cellulose. It should be understood by those skilled in the art that other stabilizing agents can be utilized which possess the desirable film properties of the present invention.

Disintegrants can aid in dissolving edible films so that the efficacy of the film can be realized sooner. Suitable disintegrants for use in the edible film of the current invention include alginic acid, microcrystalline cellulose, cross-linked cellulose, modified starches such as sodium carboxymethyl starch, as well as others known in the art. Special disintegrants known as super-disintegrants are also suitable for use in the edible film of the present invention. Super-disintegrants include crospovidone, sodium starch glycolate, and croscarmellose, which represent examples of a cross-linked polymer (polyvinylpyrrolidone), a cross-linked starch, and a cross-linked cellulosic polymer (sodium carboxymethylcellulose), respectively. These super-disintegrants are insoluble in water and most other solvents, have rapid swelling properties, and have good water uptake with high capillary action which results in fast disintegration. The components break the film down into small fragments having large surface areas which results in increased dissolution rates for the film. These components can be utilized even in low concentrations. The disintegrants or super-disintegrants can be present in amounts ranging from about 1% to about 10%, more preferably about 1% to about 5% by weight of the composition. The preferred disintegrant for use in the edible film of the current invention is croscarmellose. It should be understood by those skilled in the art that other disintegrants can be utilized which possess the desirable film properties of the present invention.

Typically, these super-disintegrants are used in a pill or tablet form with pharmaceuticals. They are ideal for use with pharmaceuticals because most are chemically inert. Some examples that use these super-disintegrants include U.S. Pat. Nos. 6,391,342; 6,177,101; and 5,629,017. One of the novel aspects of this invention is the use of these super-disintegrants for making edible films. The super-disintegrants swell extremely rapidly (high capillary action) causing a massive buildup of hydrostatic pressure which contributes to their disintegration potential.

The plasticizing agent of the present invention is utilized to improve flexibility and reduce brittleness of the edible film compositions of the present invention. The effective amount of the plasticizing agent ranges from about 0% to about 30%, more preferably about 0% to about 15% by weight of the composition. Examples of suitable plasticizing agents include, but are not limited to, glycerin, sorbitol, triacetin, monoacetin, diacetin, polyethylene glycol, propylene glycol, hydrogenated starch hydrolysates, corn syrups, derivatives thereof and combinations thereof. The preferred plasticizing agents of the present invention are sorbitol and glycerin. It should be understood by those skilled in the art that other plasticizing agents can be utilized which possess the desirable film properties of the present invention.

In a further embodiment, the edible film composition can also include an effective amount of at least one pharmaceutically active agents, an effective amount of one or more oral care agents, and an effective amount of one or more breath freshening agents.

Pharmaceutically active agents as used herein, is intended to disclose agents, other than foods, which can promote functional or structural change in the users in which they are administered. Examples of suitable pharmaceutically active agents include, but are not limited to, antimicrobial agents, anti-inflammatory drugs, anti-tussives, decongestants, anti-histamines, expectorants, anti-diarrheals, H2-antagonists, proton pump inhibitors, general nonselective CNS depressants, general nonselective CNS stimulants, antiparkinsonism drugs, narcotic-analgesics, analgesic-antipyretics, psychopharmacological drugs, derivatives thereof and combinations thereof. It should be understood by those skilled in the art that other pharmaceutically active agents can be utilized which possess the desirable film properties of the present invention.

Oral care agents can be used to help reduce oral malodor as well as act as antimicrobial agents. Examples of suitable oral care agents include, but are not limited to, caries control agents such as phosphates and fluorides, anti-plaque and anti-gingivitis agents such as cetylpyridinium chloride and triclosan, germ killing agents, and sulfur precipitating agents such as metal salts. It should be understood by those skilled in the art that other oral care agents can be utilized which possess the desirable film properties of the present invention.

Examples of suitable breath freshening agents include, but are not limited to, spearmint oil, peppermint oil, other mint oils, oil of wintergreen, zinc gluconate, citrus oils, fruit essences, clove oils, anise, menthol, eucalyptol, thymol, methyl salicylate, derivatives thereof and combinations thereof. It should be understood by those skilled in the art that other breath freshening agents can be utilized which possess the desirable properties of the present invention.

Other functional materials that can be incorporated into the edible film of the present invention include, but are not limited to, bulk fillers, mouthfeel enhancers, surfactants, sweeteners, salivary stimulating agents, binding agents, cooling agents, flavoring agents, coloring agents, fragrances, thickening agents, preservatives, derivatives thereof and combinations thereof. It should be understood by those skilled in the art that other additives can be utilized which possess the desirable film properties of the present invention.

The bulk filler agent of the present invention is utilized to reduce the oily texture of the edible film. The effective amount of the bulk filler agent ranges from about 0% to about 25% more preferably about 3% to about 15% by weight of the composition. Suitable bulk filler agents include, but are not limited to, microcrystalline cellulose, magnesium carbonate, calcium carbonate, calcium phosphate, calcium sulfate, magnesium silicate, aluminum silicate, ground lime stone, clay, talc, titanium dioxide, cellulose polymers such as wood, derivatives thereof and combinations thereof. It should be understood by those skilled in the art that other bulk filler agents can be utilized which possess the desirable film properties of the present invention.

Examples of suitable mouthfeel enhancers that can be utilized in the edible film of the present invention include microcrystalline cellulose, guar gum, xanthan gum, and carrageenan. It should be understood by those skilled in the art that other mouthfeel improvers can be utilized which possess the desirable film properties of the present invention.

Examples of surfactants (wetting agents) include sodium lauryl sulfate, mono and diglycerides of fatty acids and polyoxyethylene sorbitol esters, such as polyoxyethylene (20) sorbitan monostearate (Polysorbate 60) and polyoxyethylene (20) sorbitan monooleate (Polysorbate 80), derivatives thereof and combinations thereof. The surfactants can be present in amounts ranging from about 0% to about 10%, more preferably about 1% to about 5% by weight of the composition. The preferred surfactant for use in the edible film of the current invention is Polysorbate 80. It should be understood by those skilled in the art that other surfactants can be utilized which possess the desirable film properties of the present invention.

Sweeteners for use in the edible film of the present invention are well known in the art. Examples of such sweeteners include, but are not limited to, sucralose, sorbitol, aspartame, acesulfame, dextrose, maltose, fructose, corn syrup, other water soluble sweetening agents, other water soluble artificial sweeteners, dipeptide based sweeteners, protein based sweeteners, derivatives thereof and combinations thereof. The amount of sweetener provided will vary with the specific sweetener selected to provide the desired sweetness. The preferred sweeteners for use in the edible film of the present invention are sucralose and sorbitol. It should be understood by those skilled in the art that other sweeteners can be utilized which possess the desirable properties of the present invention.

Salivary stimulating agents can be added to the edible film of the present invention. Salivary stimulating agents can aid in dissolving of the edible film such as in the cases of users who suffer from xerostomia, a condition known as dry mouth. Suitable salivary stimulating agents for use in the edible film of the present invention include, but are not limited to, citric acid, ascorbic acid, lactic acid, malic acid, succinic acid, adipic acid, fumaric acid, tartaric acid, derivatives thereof and combinations thereof. The amount of salivary stimulating agents in the edible film range from about 0% to about 10%, more preferably about 1% to about 5% by weight of the composition. The preferred salivary stimulating agents are citric acid and ascorbic acid. Suitable binding agents include any currently available edible binding agents known within the film formulation arts. It should be understood by those skilled in the art that other salivary stimulating agents can be utilized which possess the desirable film properties of the present invention.

Suitable cooling agents include monomenthyl succinate, WS3, WS23, Ultracool II and the like. It should be understood by those skilled in the art that other cooling agents can be utilized which possess the desirable film properties of the present invention.

A variety of flavorants can be utilized within the edible film of the present invention. Suitable examples of such flavorants include, but are not limited to, artificial flavorants, flavor oils, flavoring aromatics, extracts derived from plants, leaves, flowers, fruits, derivations thereof and combinations thereof. Artificial flavors which may be suitable for use include vanilla, chocolate, coffee, cocoa, citrus oil (lemon, lime, orange, grape, grapefruit), fruit essences (apple, pear, peach, strawberry, raspberry, cherry, plum, pineapple, apricot, watermelon), derivatives thereof and combinations thereof. More generally, any flavoring or food additive described in Chemicals Used in Food Processing, publication 1274 by the National Academy of Sciences, pages 63-258, may be suitable for use. It should be understood by those skilled in the art that other flavorants can be utilized which possess the desirable film properties of the present invention.

Suitable examples of aldehyde flavorings include, but are not limited to acetaldehyde (apple), benzaldehyde (cherry, almond), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e. trans-2 (berry fruits), tolyl aldehyde (cherry, almond), veratraldehyde (vanilla), 2,6-dimethyl-5-heptenal, i.e. melonal (melon), 2-6-dimethyloctanal (green fruit), and 2-dodecenal (citrus, mandarin), cherry, grape, cinnamic aldehyde (cinnamon), citral, i.e., alpha citral (lemon, lime), neral, i.e. beta citral (lemon, lime), decanal (orange, lemon), ethyl vanillin (vanilla, cream), heliotropine, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (many types), derivatives thereof and combinations thereof. It should be understood by those skilled in the art that other aldehyde flavorings can be utilized which possess the desirable film properties of the present invention.

The amount of flavoring utilized is a matter of preference, but also depends on the flavor components that have been selected for use in the edible film. Determining the ranges necessary for each flavoring agent would be well within the capabilities of one skilled in the art without the need for undue experimentation. Overall, the amount of flavoring agents utilized in the edible film of the present invention would range from about 1% to about 40%, more preferably about 1% to about 30% by weight of the composition.

Coloring agents can be used to provide the film with a more enticing appearance. Some coloring agents useful in the edible film of the present invention include pigments such as titanium dioxide and natural food colors and dyes suitable for food, drug, and cosmetic applications. The latter coloring agents are known as FD&C dyes and lakes. A full description of all FD&C and D&C dyes and their corresponding chemical structures can be found in Kirk-Othmer Encyclopedia of Chemical Technology, Volume 5, pages 857-884. The coloring agents can be utilized in the edible film of the present invention in the range from about 0.01% to about 0.2% more preferably about 0.03% to about 0.05% by weight of the composition. It should be understood by those skilled in the art that other coloring agents can be utilized which possess the desirable film properties of the present invention.

Fragrances can be utilized to enhance the appeal of the product as necessary. Any fragrance known to those skilled in the art of edible films, such as mint oils, can be used in the present invention. Thickening agents can be utilized for enhancing the structure of the edible film. Suitable thickening agents include, but are not limited to, methylcellulose, carboxyl methylcellulose, derivatives thereof and combinations thereof. It should be understood by those skilled in the art that other thickening agents can be utilized which possess the desirable film properties of the present invention.

To improve shelf life, preservatives may be utilized in the edible film of the present invention. Suitable preservatives such as potassium sorbate and sodium benzoate may be utilized in the range from about 0% to about 5%, more preferably 0% to about 2% by weight of the compositions. It should be understood by those skilled in the art that other preservatives can be utilized which possess the desirable film properties of the present invention.

Examples of several suitable compositions are specifically set out below and in the following table.

### EXAMPLE 1

The following components are utilized to manufacture an edible film of the present invention (approximate amounts by weight of the composition): 49.61% methylhydroxypropylcellulose, 5% gum arabic, 6% microcrystalline cellulose, 2.3% cross-linked carboxymethylcellulose (croscarmellose), 5.3% glycerin, 4.3% sorbitol, 25.5% flavoring, 0.2% sucralose, 1.8% Polysorbate 80, 0.025% potassium sorbate, 0.025% sodium benzoate, and 0.04% coloring.

### EXAMPLE 2

The following components are utilized to manufacture an edible film of the present invention (approximate amounts by weight of the composition): 42.91% sodium alginate, 15% gum arabic, 4% microcrystalline cellulose, 1% cross-linked carboxymethylcellulose (croscarmellose), 5.3% glycerin, 4.3% sorbitol, 25.5% flavoring, 0.2% sucralose, 1.8% Polysorbate 80, 0.025% potassium sorbate, 0.025% sodium benzoate, and 0.04% coloring.

### EXAMPLE 3

The following components are utilized to manufacture an edible film of the present invention (approximate amounts by weight of the composition): 23.10% methylhydroxypropylcellulose, 23.10% sodium alginate, 10% gum arabic, 5% microcrystalline cellulose, 1.71% cross-linked carboxymethylcellulose (croscarmellose), 5.3% glycerin, 4.3% sorbitol, 25.5% flavoring, 0.2% sucralose, 1.8% Polysorbate 80, 0.025% potassium sorbate, 0.025% sodium benzoate, and 0.04% coloring.

### EXAMPLE 4

The following components are utilized to manufacture an edible film of the present invention (approximate amounts by weight of the composition): 24.70% methylhydroxypropylcellulose, 24.70% sodium alginate, 5% gum arabic, 6% microcrystalline cellulose, 2.51% cross-linked carboxymethylcellulose (croscarmellose), 5.2% glycerin, 4.3% sorbitol, 25.5% flavoring, 0.2% sucralose, 1.8% Polysorbate 80, 0.025% potassium sorbate, 0.025% sodium benzoate, and 0.04% coloring.

### EXAMPLE 5

The following components are utilized to manufacture an edible film of the present invention (approximate amounts by weight of the composition): 49.41% sodium alginate, 5% gum arabic, 6% microcrystalline cellulose, 2.5% cross-linked carboxymethylcellulose (croscarmellose), 5.2% glycerin, 4.3% sorbitol, 25.5% flavoring, 0.2% sucralose, 1.8% Polysorbate 80, 0.025% potassium sorbate, 0.025% sodium benzoate, and 0.04% coloring.

### EXAMPLE 6

The following components are utilized to manufacture an edible film of the present invention (approximate amounts by weight of the composition): 32.41% methylhydroxypropylcellulose, 5% sodium alginate, 12% gum arabic, 12% microcrystalline cellulose, 1.5% cross-linked carboxymethylcellulose (croscarmellose), 5.2% glycerin, 4.3% sorbitol, 25.5% flavoring, 0.2% sucralose, 1.8% Polysorbate 80, 0.025% potassium sorbate, 0.025% sodium benzoate, and 0.04% coloring.

TABLE 1
Examples of possible edible film compositions.
(approximate percent by weight)

| COMPONENT | Ex. 1 | Ex. 2 | Ex. 3 | Ex. 4 | Ex. 5 | Ex. 6 |
|---|---|---|---|---|---|---|
| Methylhydroxypropylcellulose (MHPC) | 49.61 | 0.00 | 23.10 | 24.70 | 49.41 | 32.41 |
| Sodium alginate | 0.00 | 42.91 | 23.10 | 24.70 | 49.41 | 5.00 |
| Gum Arabic | 5.00 | 15.00 | 10.00 | 5.00 | 5.00 | 12.00 |
| Microcrystalline cellulose | 6.00 | 4.00 | 5.00 | 6.00 | 6.00 | 12.00 |
| Cross-linked carboxymethylcellulose | 2.30 | 1.00 | 1.71 | 2.51 | 2.50 | 1.50 |
| Glycerin | 5.20 | 5.20 | 5.20 | 5.20 | 5.20 | 5.20 |
| Sorbitol | 4.30 | 4.30 | 4.30 | 4.30 | 4.30 | 4.30 |
| Flavors | 25.50 | 25.50 | 25.50 | 25.50 | 25.50 | 25.50 |
| Sucralose | 0.20 | 0.20 | 0.20 | 0.20 | 0.20 | 0.20 |
| Polysorbate 80 | 1.80 | 1.80 | 1.80 | 1.80 | 1.80 | 1.80 |
| Potassium sorbate | 0.025 | 0.025 | 0.025 | 0.025 | 0.025 | 0.025 |
| Sodium benzoate | 0.025 | 0.025 | 0.025 | 0.025 | 0.025 | 0.025 |
| Color | 0.04 | 0.04 | 0.04 | 0.04 | 0.04 | 0.04 |

The embodiments, as shown in Table 1, when combined with breath freshening agents can be utilized as an effective treatment of halitosis, or malodorous breath. The method of treatment comprises the steps of providing an edible film composition which includes an effective amount of at least breath freshening agent to an individual and having the individual orally consume the film composition to release breath freshening agents into the oral cavity to treat halitosis, wherein the edible film comprises methylhydroxypropylcellulose and/or sodium alginate. In an embodiment of the method, the breath freshening agent is selected from those breath freshening agents known to persons skilled in the art. The embodiment is believed to be more effective than other methods to treat halitosis because the edible film composition adheres to the oral cavity not only allowing the breath freshening agents to persist longer, but also to adhere to the creases in the oral cavity where other methods may not be effective.

The above mentioned embodiments are also effective as part of a method to treat xerostomia, also known as dry mouth. The method of treating xerostomia of the present invention comprises the steps of providing an edible film composition which includes an effective amount of at least one salivary stimulating agent to a user, wherein the edible film comprises methylhydroxypropylcellulose. In an embodiment of the method, the salivary stimulating agent is citric acid.

The above mentioned embodiments are also effective as part of a method to treat plaque or gingivitis. The method of treating plaque or gingivitis of the present invention comprises the steps of providing an edible film composition which includes an effective amount of at least one oral care agent to an individual, wherein the film composition further comprises methylhydroxypropylcellulose, and placing the film in the individual's mouth to release the oral care agent to treat plaque or gingivitis.

The above mentioned embodiments are also effective as part of a method of administering a pharmaceutically active agent to an individual. The method comprises the steps of providing an edible film composition which includes an effective amount of at least one pharmaceutically active agent to an individual, wherein the film composition further comprises methylhydroxypropylcellulose, and placing the film in the individual's mouth to release the pharmaceutically active agent.

Furthermore, the above mentioned methods allow the users to treat their conditions discreetly. The patient carries the storage container and when necessary, places one of the edible films of the present invention in their oral cavity to provide relief or treatment without disturbing or drawing the attention of others.

While the invention has been shown and described with reference to the preferred embodiments, it will be understood by those skilled in the art that various changes in form and detail may be made therein without departing from the spirit and scope of the invention as defined by the appended claims.

We claim:

1. A water-soluble film composition for oral administration comprising methylhydroxypropylcellulose, gum arabic, microcrystalline cellulose, glycerin, Polysorbate 80, sorbitol, sucralose, cross-linked carboxymethylcellulose, flavoring agent, potassium sorbate, sodium benzoate, and coloring agents.

2. The water-soluble film composition of claim 1, wherein the methylhydroxypropylcellulose is present in an amount of not more than about 70% by weight, the gum arabic is present in an amount of not more than about 20% by weight, the microcrystalline cellulose is present in an amount of not more than about 25% by weight, the glycerin is present in an amount of not more than about 15% by weight, the Polysorbate 80 is present in an amount of not more than about 5% by weight, the sorbitol is present in an amount of not more than about 15% by weight, the sucralose is present in an amount of not more than about 5% by weight, the cross-linked carboxymethylcellulose is present in an amount of not more than about 5% by weight, the flavoring agent is present in an amount of not more than about 40% by weight, the potassium sorbate is present in an amount of not more than about 0.2% by weight, and the sodium benzoate is present in an amount of not more than about 0.2% by weight.

3. The water-soluble film composition of claim 1, further comprising an amount of sodium alginate.

4. The water-soluble film composition of claim 3, wherein the sodium alginate is present in an amount of not more than 70% by weight.

5. A water-soluble film composition for oral administration comprising sodium alginate, gum arabic, microcrystalline cellulose, glycerin, Polysorbate 80, sorbitol, sucralose, cross-linked carboxymethylcellulose, flavoring agent, potassium sorbate, sodium benzoate, and coloring agents.

6. The water-soluble film composition of claim 5, wherein the sodium alginate is present in an amount of not more than about 70% by weight, the gum arabic is present in an amount of not more than about 20% by weight, the microcrystalline cellulose is present in an amount of not more than about 25% by weight, the glycerin is present in an amount of not more than about 15% by weight, the Polysorbate 80 is present in an amount of not more than about 5% by weight, the sorbitol is present in an amount of not more than about 15% by weight, the sucralose is present in an amount of not more than about 5% by weight, the cross-linked carboxymethylcellulose is present in an amount of not more than about 5% by weight, the flavoring agent is present in an amount of not more than about 40% by weight, the potassium sorbate is present in an amount of not more than about 0.2% by weight, and the sodium benzoate is present in an amount of not more than about 0.2% by weight.

7. A water-soluble film composition for oral administration comprising from about 0 to about 70% by weight of primary film forming agent, from about 0 to about 20% by weight emulsifier, from about 0 to about 25% by weight stabilizing agent, from about 0 to about 25% by weight mouthfeel enhancer, from about 0 to about 15% by weight plasticizer, from about 1 to about 5% by weight surfactant, from about 0.5 to about 5% by weight sweetener, from about 1 to about 5% by weight disintegrant, from about 1 to about 5% by weight salivating agent, from about 1 to about 40% by weight flavoring agent, and optional coloring agents.

8. The water-soluble film composition of claim 7, wherein the primary film forming agent is selected from the group consisting of methylhydroxypropylcellulose and sodium alginate.

9. The water-soluble film composition of claim 7, wherein the emulsifier comprises gum arabic.

10. The water-soluble film composition of claim 7, wherein the stabilizing agent is selected from the group consisting of gum arabic, microcrystalline cellulose, and combinations thereof.

11. The water-soluble film composition of claim 7, wherein the mouthfeel enhancer comprises microcrystalline cellulose.

12. The water-soluble film composition of claim 7, wherein the plasticizer is selected from the group consisting of glycerin, sorbitol, and combinations thereof.

13. The water-soluble film composition of claim 7, wherein the surfactant comprises Polysorbate 80.

14. The water-soluble film composition of claim 7, wherein the sweetener is selected from the group consisting of sorbitol, sucralose, and combinations thereof.

15. The water-soluble film composition of claim 7, wherein the disintegrant is selected from the group consisting of microcrystalline cellulose, cross-linked carboxymethylcellulose, and combinations thereof.

16. The water-soluble film composition of claim 7, further comprising gum arabic in an amount in the range of from about 0 to about 20% by weight.

17. The water-soluble film composition of claim 7, further comprising microcrystalline cellulose in an amount in the range of from about 0 to about 10% by weight.

18. The water-soluble film composition of claim 12, wherein the plasticiser comprises glycerin in an amount in the range of from about 0 to about 15% by weight.

19. The water-soluble film composition of claim 12, wherein the plasticiser comprises sorbitol in an amount in the range of from about 0 to about 15% by weight.

20. The water-soluble film composition of claim 13, wherein the surfactant comprises Polysorbate 80 in an amount in the range of from about 1 to about 5% by weight.

21. The water-soluble film composition of claim 14, wherein the sweetener comprises sorbitol in an amount in the range of from about 0 to about 15% by weight.

22. The water-soluble film composition of claim 14, wherein the sweetener comprises sucralose in an amount in the range of from about 0.5 to about 5% by weight.

23. The water-soluble film composition of claim 15, wherein the disintegrant comprises cross-linked carboxymethylcellulose in an amount in the range of from about 1 to about 5% by weight.

24. A method of delivering an oral care agent to the oral cavity comprising the steps of providing an edible, water-soluble film comprising at least one of either methylhydroxypropylcellulose or sodium alginate, introducing the film into an oral cavity of a user, and allowing the film to dissolve in the oral cavity of the user wherein the film comprises disintegrant, emulsifier, an oral care agent, and one or more primary film formers to prolong efficacy of the oral care agent in the film.

25. The method of claim 24, wherein the oral care agent comprises an effective amount of a suitable breath freshener.

26. The method of claim 24, wherein the edible film further comprises from about 0 to about 20% by weight stabilizing agent.

27. The method of claim 26, wherein the stabilizing agent comprises gum arabic.

28. The method of claim 26, wherein the stabilizing agent comprises microcrystalline cellulose.

29. The method of claim 24, wherein the edible film further comprises from about 0 to about 25% by weight mouthfeel enhancing agent.

30. The method of claim 29, wherein the mouthfeel enhancing agent comprises microcrystalline cellulose.

31. The method of claim 29, wherein the mouthfeel enhancing agent comprises carrageenan.

32. The method of claim 24, wherein the amount of emulsifier is in the range of from about 0 to about 25% by weight.

33. The method of claim 32, wherein the emulsifier comprises gum arabic.

34. The method of claim 24, wherein the edible film further comprises from about 1 to about 5% by weight surfactant.

35. The method of claim 34, wherein the surfactant comprises Polysorbate 80.

36. The method of claim 24, wherein the edible film further comprises from about 0.5 to about 5% by weight sweetener.

37. The method of claim 36 wherein the sweetener comprises sorbitol.

38. The method of claim 36, wherein the sweetener comprises sucralose.

39. The method of claim 24, wherein the amount of disintegrant is from about 1 to about 5% by weight.

40. The method of claim 39, wherein the disintegrant comprises cross-linked carboxymethylcellulose.

41. The method of claim 24, wherein the edible film further comprises from about 1 to about 5% by weight salivating agent.

42. The method of claim 24, wherein the edible film further comprises plasticizing agent.

43. The method of claim 42, wherein the plasticizing agent comprises glycerin.

44. The method of claim 42, wherein the plasticizing agent comprises sorbitol.

45. An edible film composition comprising primary film forming agent, emulsifier, stabilizing agent, mouthfeel enhancer, plasticizer, surfactant, sweetener, disintegrant, salivating agent, flavoring agent, and coloring agent.
